(19) United States
(12) Patent Application Publication
(10) Pub. No.: US 2024/0342000 A1
(43) Pub. Date: Oct. 17, 2024

(54) METHOD OF USE OF SNORING AND OBSTRUCTIVE SLEEP APNEA PREVENTION DEVICE

(71) Applicants: CANER KARACEPER, OTTAWA (CA); CEM KARACEPER, OTTAWA (CA); ANIDA DZAFIC, OTTAWA (CA); PRAKASH CRJ NAIDU, OTTAWA (CA)

(72) Inventors: PRAKASH CRJ NAIDU, OTTAWA (CA); CANER KARACEPER, OTTAWA (CA); CEM KARACEPER, OTTAWA (CA); ANIDA DZAFIC, OTTAWA (CA)

(21) Appl. No.: 18/630,344

(22) Filed: Apr. 9, 2024

Related U.S. Application Data
(62) Division of application No. 17/824,897, filed on May 26, 2022.

(51) Int. Cl. A61F 5/56; A61C 13/00

(52) U.S. Cl. CPC A61F 5/566; A61C 13/0019

(57) ABSTRACT

A method of use of an oral appliance (OA) made as add-on optional feature in a denture of a user with or without implants to facilitate adjustable mandibular advancement assisting in partially or fully reducing snoring and thus ameliorating obstructive sleep apnea (OSA). Steps of the method of use include obtaining intra-oral information necessary and adequate for manufacture of a primary denture set for chewing purposes and using the same information for manufacture of a simpler, lighter secondary denture set that may be offered to a patient ordering a primary denture set, at a discounted price that may serve the purpose of cosmetic function or anti-snoring function or both.

### CROSS-REFERENCE TO RELATED APPLICATIONS

This is a divisional patent application that incorporates in by reference the entire contents of the Parent application Ser. No. 17/824,897 (Confirmation No. 7745) filed dated 26 May 2022.

### STATEMENT REGARDING FEDERALLY SPONSORED RESEARCH OR DEVELOPMENT

The work on which the disclosure of this application is based is not funded by any Federally Sponsored Research or Development in the United States of America.

### BACKGROUND OF THE INVENTION

### Field of the Invention

The present invention pertains to oral appliances for the prevention of snoring, sleep apnea, or similar other conditions, more particularly to an oral appliance that can be used by those who wear dentures, methods of their manufacture and use.

### Description of the Related Art

It is well known and documented that those individuals suffering from sleep-related breathing disorders such as sleep apnea exhibit moderate to excessive snoring which is often ignored or neglected without any preventive or curative measures. It is also well known that this neglect is causing an increased risk resulting in more severe health problems later on such as stroke, atrial fibrillation, hypertension, chronic heart failure, excessive sleepiness in daytime causing driving accidents or equipment operation accidents, and a variety of ailments e.g. diabetes, depression, among others including the fact that an obstructive sleep apnea (OSA) episode can be life threatening; however, 80 percent of the cases of moderate and severe obstructive sleep apnea is likely undiagnosed (Source: American Sleep Apnea Association: Sleep Apnea Information for Clinicians-A very short course on sleep apnea; Hu, Pub. No. US 2020/0129278 dated Apr. 30, 2020). Studies have shown, for instance, that individuals demonstrating snoring are most likely to be suffering from OSA that may lead to one or more of the aforementioned health problems. OSA is characterized by periodic and repetitive collapse of the upper airway during sleep, the frequency of the same is specified as apnea and hypopnea index (AHI) that shows the average number of apneas and hypopnea per hour of sleep. Health care professionals use the terms of respiratory disturbance index (RDI), and AHI interchangeably. While apnea episodes entail total stoppage or pause in breathing typically for 20 to 40 seconds, hypopnea episodes entail typically shallow or partial breathing accompanying 30% or greater reduction in air flow lasting for 10 seconds or longer with an associated greater desaturation in the person's oxygen levels exceeding 4% often resulting in fragmentation of sleep or arousal. The AHI is calculated by dividing the sum of apneas and hypopneas by the number of hours of sleep. AHI values are categorized as follows: severe if more than 30 episodes per hour, moderate if episodes ranging from 15 to 30 per hour, and mild if 5 to 15 episodes per hour. These episodes of reduced airflow are typically caused by the relaxation of the tongue and pharyngeal walls to varying degrees during the several stages of sleep wherein the musculature supporting these tissues relaxes, therefore, as air is inhaled, the tongue and posterior walls of the pharynx collapse, causing snoring that is in fact vibration noise caused by partial or complete obstruction of the airway. The episodes may cause several undesirable conditions such as reduced oxygen level in the blood (hypoxemia), elevated circulating carbon dioxide (hypercapnia), and arousal from sleep to re-establish a stable airway.

A rigorous study published in 2019 in Lancet reports (Markets and Markets. Sleep Apnea Devices Market, Report Code: MD 1181, Published Date: August 2021) that nearly 1 billion adults aged 30-69 years worldwide could have OSA, and the number of people with moderate to severe OSA, for which treatment is generally recommended, is estimated to be almost 425 million. Major part of the actual market remains untapped or not served as a large number of patients remain undiagnosed (Adam V Benjafield, Najib T Ayas, Peter R Eastwood, Raphael Heinzer, Mary S M Ip, Mary J Morrell, Carlos M Nunez, Sanjay R Patel, Thomas Penzel, Jean-Louis D Pépin, Paul E Peppard, Sanjeev Sinha, Sergio Tufik, Kate Valentine, and Atul Malhotra. Estimation of the global prevalence and burden of obstructive sleep apnoea: a literature-based analysis. Lancet Respir Med. 2019 August; 7 (8): 687-698) as they do not even know the condition and risks because the symptoms arise only while the person is in sleep and often does not believe when very limited close individuals, such as a bed partner may dare to point out, more due to noise creation annoyance such as snoring or in some cases combined with grinding of teeth (known as bruxism) and seldom due to genuine health concerns of the sufferer. The highest number of sufferers are in China, USA, Brazil, and India in decreasing order, with USA estimated to have more than 54.1 million with AHI of 5 or more events per hour; and more than 23.6 million with AHI of 15 or more events per hour. Some estimates are even higher, for example, Ramabadran reported in U.S. Pat. No. 11,147,506 dated Oct. 19, 2021 titled “Sleep diagnostic system and method” that close to 65 million American adults suffer from sleep disordered breathing conditions where 1 in 5 adults has obstructive sleep apnea or central sleep apnea conditions and contemplated that out of this large number of estimated sufferers, probably just 1 in 15 adults are properly screened, tested and then treated for this condition, leaving a substantially huge untested and thus unattended population. Canada has a substantial number of affected persons: more than 4.7 million with AHI of 5 or more events per hour; and more than 0.9 million with AHI of 15 or more events per hour. The extent and nature of the problem has attracted ongoing discussions on need for change in the way in which the diagnosis and treatment is currently funded by public health and private insurance coverage (Sachin R. Pendharkar, Marcus Povitz, Nick Bansback, Charles F. P. George, Debra Morrison, Najib T. Ayas for the Canadian Sleep and Circadian Network. Testing and treatment for obstructive sleep apnea in Canada: funding models must change. CMAJ 2017 Dec. 11; 189: E1524-8. doi: 10.1503/cmaj.170393).

While severe sleep-related breathing disorders may require Continuous Positive Airway Pressure (CPAP) therapy, less severe sleep-related breathing disorders may be treated with other therapies or devices, such as an Oral Appliance (OA). Generally, an oral appliance includes either a single mono-block device to impart forward advancement of the user's lower jaw (mandible) relative to the user's upper jaw (maxilla); or alternatively a duo-bloc device wherein upper and lower dental trays are coupled together in such a way so as to achieve similar mandibular advancement. Accordingly, an OA is generally referred to as a Mandibular Advancement Device (MAD) or a Mandibular Advancement Splint (MAS). Unblocking of a user's airway during sleep is achieved by the forward advancement of the mandible that helps prevent the soft tissue of the tongue and the throat from collapsing into the airway passage. In an OA, it is important to achieve the correct amount of forward mandibular advancement. For example, the soft tissue of a user's tongue and throat may not be prevented from collapsing into the user's airway and thus they may continue to Suffer from a sleep-related breathing disorder if the forward mandibular advancement imparted by the OA is inadequate. Conversely, a user may experience undesirable discomfort in the mandibular musculature, tooth pain, or even more serious side effects such as temporomandibular joint (TMJ) pain if the forward mandibular advancement imparted by the OA is excessive (as reported, for example, by Marklund, M.; Franklin, K. A. Long-term effects of mandibular repositioning appliances on symptoms of sleep apnoea. J. Sleep Res. 2007, 16, 414-420; and Pliska, B. T.; Nam, H.; Chen, H.; Lowe, A. A.; Almeida, F. R. Obstructive sleep apnea and mandibular advancement splint: Occlusal effects and progression of changes associated with a decade of treatment. J. Clin. Sleep Med. 2014, 10, 1285-1291). In extreme cases, the discomfort may cause the user to stop wearing the OA all together, in many other cases, it is reported that undesirable arousal from sleep may occur.

Some oral appliances have been disclosed that include mechanisms for adjusting the amount of mandibular advancement provided by the oral appliance. These are called adjustable or titratable devices.

Halstrom describes in U.S. Pat. No. 5,365,945 dated Nov. 22, 1994 titled “Adjustable Dental Appliance for Treatment of Snoring and Obstructive Sleep Apnea” an appliance that consists of an upper bite block conforming to the patient's maxillary dentition, a lower bite block conforming to the patient's mandibular dentition, and a connecting assembly secured to an anterior region of the upper and lower bite blocks for adjustably or in a titratable manner coupling the upper and lower bite blocks together.

Lowe describes in WO1995019746 dated 27 Jul. 1995 titled “Mandible repositioning appliance for snoring prevention” a device formed by an upper bite block and a lower bite block interconnected by an extendable connector including a posterior section connected to the rear portion of the upper bite block and an anterior section connected to the front portion of the lower bite block and an adjustable threaded element interconnection between the anterior and posterior sections affecting the relative positions.

Fenton describes in U.S. Pat. No. 5,499,633 dated Mar. 19, 1996 titled “Anti-snoring Device with Adjustable Upper and Lower Relational Members” an anti-snoring device that comprises an upper member for receiving upper teeth of the user, which is joined in a spaced relationship to a lower member for receiving lower teeth of the user. The lower member is selectively positioned with respect to the upper member such that the lower member, by receiving the lower teeth of the user, moves the lower jaw of the user to a position that is forward of its “normal’ resting or biting position. This forward movement of the lower jaw is claimed to significantly open the airway of the user, thereby alleviating, reducing, or eliminating snoring.

Kidd and Lane describe in U.S. Pat. No. 5,829,441 dated Nov. 3, 1998 titled “Customizable Dental Device for Snoring and Sleep Apnea Treatment” a mandible extension dental device which includes adjustable upper and lower arch trays and coupling means designed to pull the lower jaw of a user forwardly during Sleep, so as to minimize snoring and mild apnea conditions. The device is intended to be rapidly fitted in the field without the need for the services of a dentist.

Halstrom describes in U.S. Pat. No. 5,868,138 dated Feb. 9, 1999 titled “Dental Appliance for Treatment of Snoring and Obstructive Sleep Apnea” yet another configuration of a dentally retained intra-oral appliance worn at night for treatment of snoring and obstructive sleep apnea.

Strong describes in U.S. Pat. No. 6,526,982 dated Mar. 4, 2003 titled “Anti-snoring Device and Method of Making Same” an anti-snoring device that has maxillary and mandibular bite forms with outwardly extending pivots which are mounted to the bite forms by frameworks which are at least partially embedded in the bite forms supposedly able to retain the pivots securely in place even in the face of extensive bruxing.

Palmisano describes in U.S. Pat. No. 6,536,439 dated Mar. 25, 2003 titled “Apparatus and methods for treatment of conditions including obstructive sleep apnea and snoring” a maxillary expansion device that is fitted to teeth of the upper jaw, and the maxilla is expanded by operation of a jack screw, such that, after the maxilla stabilizes, the minimum cross-sectional area of the nasal cavity increases, reducing nasal airway resistance resulting in curing, or ameliorating, obstructive sleep apnea and/or snoring.

Palmisano describes in U.S. Pat. No. 6,604,527 dated Aug. 12, 2003 titled “Mandibular Advancement Device” a custom made device made for each user from a desired cast of the user's jaw having a lower bite block, with a pair of upwardly extending flanges, and an upper bite block, with a pair of downwardly extending flanges; the flanges each carry engagement surfaces. When the bite blocks are fitted to the jaws of a user during sleep, the lower and upper engagement surfaces engage and cause anterior advancement of the lower jaw from the reflex path of opening and maintain that engagement and advancement whilst permitting movement, up to the normal range of jaw opening.

Palmisano and Mehta describe a version of the above in EP 1094761 published dated 29 Oct. 2008. Bedford discloses some updates and improvements over Palmisano described above, in US 2018/0360646 dated Dec. 20, 2018 and in AU 2021250941 dated Oct. 15, 2021 titled “A Mandibular Advancement Device”. Introduction of a ratchet mechanism to effect the mandibular advancement along with the lower and upper parts being formed from relatively hard lower and upper shells, relatively soft lower and upper linings, and heat formable lower and upper inner linings therebetween appear to be the newly claimed innovative steps.

Scarberry et. al. describe in U.S. Pat. No. 8,205,617 dated Jun. 26, 2012 titled “Oral Appliance for Treatment of Snoring and Sleep Apnea” an oral appliance system that comprises an upper tray and a plurality of lower trays. The upper tray is adaptable to conform to a user's maxillary dentition and each of the lower trays is adaptable to conform to the user's mandibular dentition. The lower trays are structured to engage the upper tray and each of the lower trays is structured to impart a different fixed amount of mandibular advancement and/or different amount of vertical height.

Kopp describes in U.S. Pat. No. 8,640,705 dated Feb. 4, 2014 titled “Anti-Snoring Device” an arrangement for mandibular advancement wherein elongated holes and so called S-shaped design of a pair of protrusion ties in conjunction with U-shaped upper and lower jaw bars are claimed to provide elasticity and thus spring action.

Fallon, Jung, and Fallon describe in U.S. Pat. No. 8,833,374 dated Sep. 16, 2014 titled “Intra-oral Mandibular Advancement Appliance” a mandibular advancement device wherein upper and lower tray assemblies having shapes against which the teeth of user's upper and lower jaws are seated respectively may be adjusted and locked in different positions respective with each other. A tongue rest is also provided to prevent tongue from falling across and blocking the patient's airway.

Petelle and Fleury describe in U.S. Pat. No. 9,545,330 dated Jan. 17, 2017 titled “Intraoral orthosis, a method of fabricating such an orthosis, and a method of adjusting it” an intraoral orthosis for treating snoring and sleep-apnea syndrome that includes two shells connected together by an adjustable connection device, the adjustable connection device including two notched tabs that are arranged respectively on either side of one of the shells, each notched tab co-operating in adjustable manner with a respective adjustment housing, arranged correspondingly on either side of the other of the shells, the adjustment housings being molded integrally with the other shell, each adjustment housing being connected to the other shell by a foldable support structure. A version of this was earlier described in U.S. Pat. No. 9,095,454 dated Aug. 4, 2015 titled the same.

Kim et. al. describe in U.S. Pat. No. 9,949,868 dated Apr. 24, 2018 titled “Mandibular Advancement Device” a configuration wherein relative positions of upper and lower fins on upper and lower splints enable desired mandibular advancement, claiming manufacturing a set of at least two devices following a process for digital design based on obtaining electronic data on the shape of user's dentition as the invention.

Baratier and Palomino describe in U.S. Pat. No. 10,363,160 dated Jul. 30, 2019 titled “Mandibular Repositioning Device” an intra-oral device comprising an upper splint structured to engage with one or more teeth on user's maxilla, a lower splint structured to engage with one or more teeth on user's mandible, a pair of lateral connecting rods (each connecting rod has a first rod end that connects to the lower splint and a second rod end that connects to the upper splint), and the connecting rods are configured to maintain the mandible in an advanced position relative to the maxilla.

Veis and Ataii describe in U.S. Pat. No. 10,363,161 dated Jul. 30, 2019 titled “Sleep Apnea Oral Appliance for Use During Orthodontic Treatment” a sleep apnea oral appliance in which a pair of appliance trays are worn over orthodontic trays coming in contact with maxillary dentition and mandibular dentition respectively that may comprise a series of different configurations in order to change the position of a user's teeth and/or the shape of the user's jaw.

Fallon describes in U.S. Pat. No. 10,849,783 dated Dec. 1, 2020 titled “Full Movement Jaw Advancement Oral Appliance to Reduce the Effects of Snoring and/or Sleep Apnea” an oral appliance that includes an upper arch tray assembly against which the user's upper teeth carried are received, a lower arch tray assembly against which the user's lower teeth are received, and an intermediate guide post support tray that is connected at the bottom thereof to the lower arch tray assembly and at the top thereof to the upper arch tray assembly by an upper arch tray retaining post that extends therebetween.

Mortadi describes in U.S. Pat. No. 10,888,451 dated Jan. 12, 2021 titled “Oral Device for Preventing Sleep Apnea and a Method of Manufacturing” an oral device that provides an upper plate and lower plate, each adapted to retain the biting surface of the respective set of maxilla and mandible teeth, wherein a pair of S-shaped curved rods pivotally interconnecting both plates are adapted for maintaining an operable spaced apart relationship therebetween.

Radmand describes in U.S. Pat. No. 11,191,663 dated Dec. 7, 2021 titled “Oral Appliance and Kit for Treatment of Sleep Apnea” an oral appliance for the treatment of obstructive sleep apnea that includes a mouthpiece embedded with a variety of sensors and a stimulator configured for being received in an oral cavity of a user.

Kopelman describes in U.S. Pat. No. 11,207,208 dated Dec. 28, 2021 titled “Systems and Methods for Positioning a Patient's Mandible in Response to Sleep Apnea Status” three different optional embodiments wherein a rotor element, or a linear advancement structure, or a tether element winding and unwinding are deployed along with use of actuator to achieve relative advancement of the bottom part against the upper part.

These and similar other oral appliances, however, are all directed for users with natural teeth as the devices are meant to be worn over the mandibular dentition and maxillary dentition. Moreover, they are expensive, complicated to manufacture, and have adjustment mechanisms that are difficult to operate. Furthermore, the OAs currently available that incorporate adjustment mechanisms tend to be bulky, thereby adversely impacting the user's comfort level, additionally, these known OAs are difficult to properly fit, do not allow sufficient vertical or horizontal movement of the jaw when worn, and are not flexible enough to allow to be suitable for those who generally wear standard ordinary, or implant retained dentures also known as overdentures in the art. Accordingly, a need exists for a more specific oral appliance for the avoidance of snoring and thus possible preventive measures or treatment of OSA that can provide a solution for this unaddressed user market segment while overcoming at least partially the aforementioned limitations and other shortcomings associated with known oral appliances.

### BRIEF SUMMARY OF THE INVENTION

### Statement of the Object of the Invention

Wearable oral device solution OAs for avoiding snoring and thus possibly OSA such as Mandibular Advancement Devices (MADs) are typically very similar to dentures and can be suggested by a dentist or even a denturist for use avoiding the logistical complexity of sleeping overnight in a lab for the Level 1 test required for a physician prescribed intervention. Broad market under sleep disorders includes diagnosis at different levels of complexity (Level 1, Level 2, Level 3 and Level 4 in decreasing order of complexity) interventions followed by CPAP as a directly physician recommended primary option and subsequently moving on to an OA for mainly those who do not tolerate or do not prefer CPAP. Considerable number of users of CPAP machines have expressed that there are many problems with using them, for example, difficulty getting the right style and size of mask, difficulty in getting used to wearing the device during sleep, difficulty tolerating forced air, dry nose and/or mouth, feeling claustrophobic, mask leak, skin irritation or pressure sores, difficulty falling asleep, and many such inhibitions. The difficulties inherent to CPAP machines lead to a high failure rate perhaps as much as 50% among patients using them (Hu, Pub. No. US 2020/0129278 dated Apr. 30, 2020). This current approach of CPAP as first line of OSA treatment that too only after the situation becomes serious appears to be the main cause for the massive number of undiagnosed sufferers (Benjafield et. al., Lancet, 2019).

The niche area we are addressing initially through this invention disclosure is to achieve relatively small but focussed market servicing in the senior citizens (considered as 60+ years in some countries, but may vary from country to country such as lower or higher such as 65+ years in some countries) and near senior citizens (for example 50+ to 55+ years age up to senior citizen age) market who anyways need full or partial dentures irrespective of potential snoring or OSA. According to several estimates quoting the Journal of Prosthetic Dentistry, 37.9 million Americans were estimated to require dentures in 2020 (Douglass et. al. “Will there be a need for complete dentures in the United States in 2020?”, J. Prosthet Dent. 2002 Jan.; 87 (1): 5-8. doi: 10.1067/mpr.2002.121203). That's nearly 9% of the United States population. As Canadian population is approximately 0.11 of the USA population (Simon Fraser University. Population Comparison of Canada, United States, and Mexico: Profile Report, World Population), assuming similar North American demographics, the estimated denture users in Canada are 4.17 million, adding up the potential users of the disclosed invention to about 42 million. By providing features in these denture devices that either embedded integrally or as add-on optional accessories augment dentures into preventive snoring avoidance and thus a first line of means to avoid early OSA onset or suffering, we would be providing a pioneering solution not available in the market currently.

The strategy we have planned through this invention disclosure to address the needs of this niche but substantial market segment is to offer precautionary and preventive anti-snoring option, that addresses the annoyance of snoring noise creation, but also safeguards one from OSA, whether or not diagnosed. It may be noted that all currently available MADs in market described in the earlier section of this disclosure are worn over the natural teeth, and thus the field is wide open without any competitive product that can be worn by this niche market segment.

The only option that comes closest to the needs of this niche market that is available currently for full denture wearers is from an Australia headquartered company offered as Somnomed® Edentulous™ costing a considerable amount. However, this product is not part of a denture or cannot be worn over denture; it is an additional product that needs the user to remove their regular denture and to obtain and wear this expensive device separately. Another constraint is that the edentulous option can be offered only from the SomnoDent® Flex or SomnoDent® Classic or SomnoDent® MAS for users with no upper teeth (in the maxilla), which implies that existence of lower teeth (in the mandible) is necessary. In general, it is recommended that as a pre-requisite, a user should have six lower teeth (3-3) in the mandible (as per product brochure SomnoDent-Edentuluos-MAS-Brochure.pdf). Therefore, we can conclude from this brief of the statement of the object of the invention that filling a much desirable gap in the field by providing a solution to a substantial number of sufferers from snoring and obstructive sleep apnea would be a first-time introduction of such an option for people without full or partial natural teeth.

### Gist of the Invention

The preferred embodiment of disclosed invention provides an add-on optional feature in a denture of a user to facilitate adjustable or titratable mandibular advancement assisting in partially or fully reducing snoring and thus ameliorating obstructive sleep apnea. The add-on optional feature in the denture set maybe provided in either a regular denture without implants, such as Removable Partial Dentures (RPDs), Removable Full Dentures (RFDs); or in any of the Implant Supported Dentures (ISDs), viz. single tooth dental implant, multiple-tooth on implant-supported bridge, and multiple-tooth on implant-retained denture. Dentures are also called denture prosthesis.

According to one aspect of the invention, the denture set may be provided with an optional set of pins or other means to hold a mandibular part of a denture in an advanced position relative to a maxillary part of the denture using a set of straps of different lengths. Additionally, and optionally, the straps may have a slit or recess to allow relative movement between the mandibular and maxillary parts of the denture.

Courting to another aspect of the invention, optionally, but in addition, the straps may be made of light weight flexible bio-compatible material such as Nylon-12 or a stretchable bio-compatible elastomer allowing relative movement between the mandibular and maxillary parts of the denture in multiple directions of 3D space, viz. forward-backward, upward-downward, leftward-rightward. The straps may be manufactured by several alternative means such as molding, 3D printing, blanking or stamping or other methods that may be available in the state-of-the-art.

Courting to yet another aspect of the invention, optionally, but in addition, the straps may be made of multi-layer hybrid construction wherein a stronger material such as Kevlar® (DuPont®, Wilmington, Delaware, USA) cord is the core to provide strength over which bio-compatible material such as Nylon-12 is coated allowing a combination of both strength as well as flexibility in the relative movement between the mandibular and maxillary parts of the denture in multiple directions of 3D space, viz. forward-backward, upward-downward, leftward-rightward. The multi-layer construction may be achieved by several alternative manufacturing means such as molding, 3D printing, or other methods that may be available in the state-of-the-art.

According to an additional but optional aspect of the invention, the preliminary impression of a patient's mouth obtained by a dentist or denturist by applying pressure to the soft tissues to simulate biting force and extending the borders of the mold to adjacent toothless areas to allow the dentures to better adapt to the gums, usually done in wax to prepare a diagnostic cast or a final cast made of gypsum or other suitable material may be gainfully utilized for the dual purpose of making a denture set as well as a splint set for mandibular advancement.

According to another additional but optional method of use of the invention, the configuration of the patient's denture available with a dentist or denturist may be gainfully utilized for the add-on purpose of making an optional light-weight splint set for mandibular advancement providing a preventive measure for avoiding snoring and thus ameliorating obstructive sleep apnea for those willing to wear denture during sleep.

According to yet another additional but optional embodiment of the invention, an approximate configuration of a patient's natural teeth or denture partially may be adequate to be utilized for the purpose of making a splint set for mandibular advancement deploying a clamp-on approach instead of a complete-fit approach providing a preventive measure for avoiding snoring and thus ameliorating obstructive sleep apnea.

In summary, the disclosed invention of snoring and obstructive sleep apnea prevention device offers considerable improvement and advantages over those described in prior art in general, as an optional accessory for dentures in particular.

### BRIEF DESCRIPTION OF THE DRAWINGS

FIG. 1A presents a photographic view of a typical conventional full denture set.

FIG. 1 shows an overall assembled view of an embodiment of the invention having a maxillary full denture, a mandibular full denture, the two connected by a pair of straps.

FIG. 1B presents a view of a typical strap connecting a maxillary denture, and a mandibular denture.

FIG. 1C shows a sectional view of the FIG. 1B.

FIG. 2A shows a representative set of straps of different lengths to enable different distances of mandibular advancement.

FIG. 2B, 2C, 2D show alternative embodiments of configurations of the straps and shapes of pin heads that enable advantages in ensuring securing and ease of change of straps.

FIG. 2E shows a sectional view of a typical multi-layer strap configuration with a stronger cord material reinforcing as core to enable improved durability with flexibility.

FIG. 3 shows a punch and die set for blanking process as an optional manufacturing process for straps to enable economy of scale.

FIG. 4 shows an overall assembled view of an embodiment of the invention having a typical ISD set comprising of a maxillary part, a mandibular part, and a pair of straps connecting the maxillary and mandibular parts to enable desired mandibular advancement.

FIG. 5 shows the schematic diagram for an optional additional production and business process offering a secondary discounted priced unit to a patient ordering a primary denture generating add-on revenue.

### DETAILED DESCRIPTION OF THE INVENTION

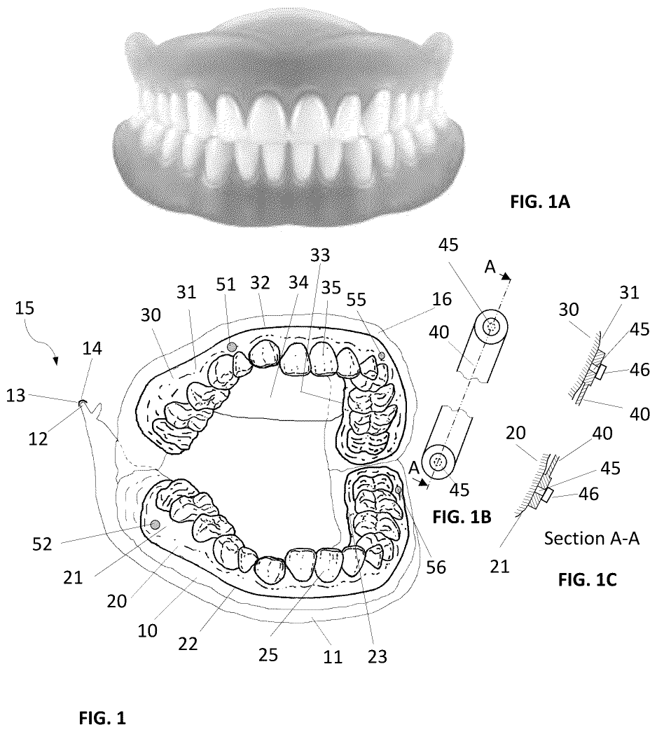

An overall illustration of a conventional full denture set is presented in a photographic view of a typical set in FIG. 1A. The set is well known in the art as upper or maxillary denture and lower or mandibular denture. Each denture has a denture base, a denture flange, denture borders and denture teeth.

FIG. 1 illustrates a configuration of mandibular and maxillary dentures in relation with the lower and upper jaws of a user respectively, pointing out the locations of different anatomical parts of interest in view of the disclosed inventive aspects for fixation of straps to realize mandibular advancement to mitigate snoring or OSA. The jaws of user are shown in an open state to be able to view the internal parts of oral cavity of the user. The lower jaw has lower gum (also called gingiva) part 10 placed over a bone structure 11 in the mandible. The bone structure extends to the back as a Temporomandibular Joint (TMJ) 15 comprising of parts known in the art as condyle 12, fossa 13, and disc 14. The mandibular denture 20 is placed over the lower gum 10 in a reasonably tight but comfortable fit and remains retained during day-to-day activities of the user such as speaking, chewing, etc. In a standard denture that does not use any implants to secure the denture to the mandible, the shape and fit between the mandible and the mandibular denture serve the purpose of retention.

The user is generally provided with options for increased levels of retention, for example, by inserting or applying between denture 20 and gum 10 bio-compatible human use safe glue materials claiming several characteristics such as being Zinc free, impervious to water, providing soft cushion, quality seal from food particles, ooze-control, use of natural ingredients like gum wax, beneficial herbs, and vitamins. Several glue products are available, for example, Effergrip® denture adhesive cream (manufacturer: Prestige Consumer Healthcare, Irvington, New York; Source: Amazon® USA), Fixodent® Ultra Max Hold Dental Adhesive (Procter & Gamble®, Cincinnati, OH, USA), SECURE® Sensitive Adhesive (Cutting Edge International, LLC, Los Angeles, CA, USA), Instant Smile™ Secure Fit Adhesive (Billy Bob Products Inc., Hardin, IL, USA), Super Poligrip® Denture and Partials Adhesive Cream (Glaxo Smith Kline, Philadelphia, PA, USA), Corega® Denture Adhesive Cream (Glaxo Smith Kline, Bulgaria, Source: Amazon®, USA), Y-Kelin Denture Adhesive Cream (Anhui Greenland Biotech Co. Ltd., Bengbu, Anhui, China), DenSureFit® Lower Denture Reline Kit (OTC Dental Inc., Vancouver, WA, USA), and from many other manufacturers or sources.

The use of glue between denture and gum is found to achieve good average retention force in compliance with reported research, for example, in the range of 16.66±7.32 lbs for milled denture bases and in the range of 12.19±6.15 lbs for the conventional heat polymerized denture bases (AlHelal, Abdulaziz Abdullah, “Comparison of Retention between Milled and Conventional Denture Bases: A Clinical Study” (2016), Loma Linda University Electronic Theses, Dissertations & Projects, 323). Our experimental tests have shown that 3D printed dentures provide better or at least comparable retention force relative to milled denture bases. We have found that this retention force is adequate to withstand the mandibular advancement force reported in the art as approximately 1 to 1.2 Newtons per millimeter of mandibular advancement (for example, 1.18 Newtons per millimeters as per J Cohen-Levy, B Petelle, J Pinguet, E Limerat, B Fleury, Sleep Breath, 2013 May; 17 (2): 781-9. doi: 10.1007/s11325-012-0765-4. Epub 2012 Sep. 11).

The mandibular denture 20 comprises of an exterior wall or flange 21, an exterior ridge or border 22, and an interior ridge or border 23. A cavity known in the art as trough is formed between the exterior and interior walls that fits over the lower gum 10. A set of teeth 25 that substitute natural teeth known in the art with nomenclature (starting from back or TMJ side, a set of teeth-one on left and one on right sides) Second Molar, First Molar, Second Premolar, First Premolar, Canine or Cuspid, Lateral Incisor, and Central Incisor (front most teeth set) are embedded symmetrically in the mandibular denture in a U-shaped configuration. This is considering the prevailing trend of not having Third Molar or Wisdom teeth at the back most location, like the natural teeth set where their extraction is recommended by dentist profession, resulting in a total fourteen teeth in the mandibular denture.

Analogous to the mandibular denture 20, the maxillary denture 30 comprises of an exterior wall or flange 31, an exterior ridge or border 32, and an interior ridge or border 33. A cavity known in the art as trough is formed between the exterior and interior walls that fits over the upper gum 16. Generally, an impression of upper palate of user's oral cavity is obtained to provide a bridge between the left and right sides of interior walls in the maxillary denture. This bridge part known as Palate 34 generally is in close contact with the user's upper palate helping in improved retention of the maxillary denture. The maxillary denture 30 is placed over the upper gum 16 in a reasonably tight but comfortable fit and remains retained during day-to-day activities of the user such as speaking, chewing, etc. In a standard denture that does not use any implants to secure the denture to the maxilla, the shape and fit between the maxilla and the maxillary denture along with palatal contact serve the purpose of retention. The user is generally provided with options for increased levels of retention, for example, by inserting or applying between denture 30 and gum 16 bio-compatible human use safe glue materials claiming several characteristics as mentioned earlier in the context of mandibular denture. A set of teeth 35 that substitute natural teeth known in the art with nomenclature (starting from back or TMJ side, a set of teeth-one on left and one on right sides) Second Molar, First Molar, Second Premolar, First Premolar, Canine or Cuspid, Lateral Incisor, and Central Incisor (front most teeth set) are embedded symmetrically in the maxillary denture in a U-shaped configuration. This is considering the prevailing trend of not having Third Molar or Wisdom teeth at the back most location, like the natural teeth set where their extraction is recommended by dentist profession, resulting in a total fourteen teeth in the maxillary denture.

A location 51 on the user's right-side wall 31 of the maxillary denture 30, and location 52 on the user's right-side wall 21 of the mandibular denture 20 exemplify the locations of connection of pins 45 illustrated in FIG. 1B and FIG. 1C. Similarly, a location 55 on the user's left-side wall of the maxillary denture 30, and location 56 on the user's left-side wall of the mandibular denture 20 exemplify the locations of connection of pins 45 illustrated in FIG. 1B and FIG. 1C. The pins may be integrated structurally with respective denture body while manufacturing the same by molding, milling, 3D printing or additive manufacturing or other fabrication methods. Alternatively, other approaches also may be deployed such as affixing the pins into the denture bodies by adhesion, fitting into receptor holes, or by screwing into receptor threaded holes by having matching threads in the pins, or a combination of such approaches. FIG. 1B illustrates a view of a strap 40 assembled with the dentures using the pins 45 as viewed from the front of the walls of the dentures.

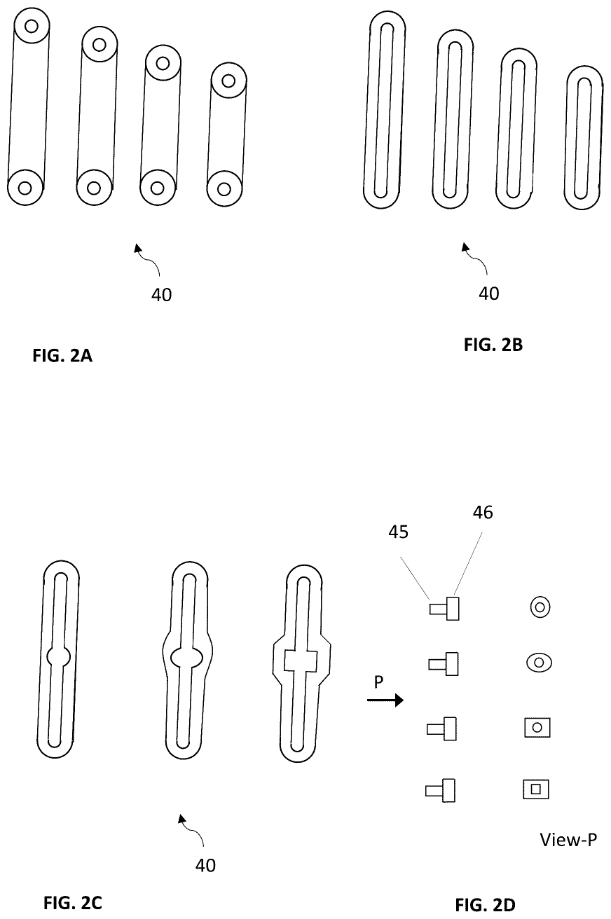

FIG. 1C illustrates a sectional view of the assembly shown in FIG. 1B wherein the strap 40 is retained or fixed on the maxillary denture 30 and mandibular denture 20 using a pair of pins 45 that have pin heads 46. The pins and pin heads are not sectioned in the section A-A view shown in FIG. 1C. The pin heads 46 may be an integral part of the pins 45 in an embodiment of the invention. The pin heads 46 may be attached to the pins 45 in another optional embodiment of the invention by means such as threads, snap fit, and other methods. The shapes of the pins and the pin heads are shown as circular cylindrical in the embodiment illustrated in the FIGS. 1B and 1C, however, those skilled in the art may appreciate that there are several other conceivable shapes possible, and all such shapes are included in the spirit of this invention. The gainful deployment of a few of those shapes are illustrated in FIG. 2D and described in the following paragraphs.

FIG. 2A illustrates a set of 4 different lengths of strap 40. The set of different lengths enables customization of the amount of mandibular advancement that may work most effectively for a particular user. Typically, the relative positions of pins 45 fixed on the mandibular denture 20 and maxillary denture 30 is such that use of shortest strap results in maximum mandibular advancement, whereas the use of longest strap results in minimum mandibular advancement. Different strap lengths may be tried between these to optimise the amount of mandibular advancement best suited for a particular user considering factors such as clearance of airway passage, and comfort of use including reduction or management of side effects such as temporomandibular joint (TMJ) pain. Optionally and preferably, the configuration of a strap 40 illustrated in the Figures has a relatively thicker boss area surrounding the pin hole to provide better strength and thus resistance to tear due to the mandibular advancement force reported in the art as approximately 1 to 1.2 Newtons per millimeter of mandibular advancement (for example, 1.18 Newtons per millimeters as per J Cohen-Levy, B Petelle, J Pinguet, E Limerat, B Fleury, Sleep Breath, 2013 May; 17 (2): 781-9. doi: 10.1007/s11325-012-0765-4. Epub 2012 Sep. 11, as quoted earlier). Those skilled in the art may appreciate that there may be other number of straps in a set ranging from 2 to 10 or more or even a single strap optimised a priori before handing over to a user, and all such possibilities are within the scope and spirit of this invention disclosure.

FIG. 2B illustrates a set of 4 different lengths of another optional configuration of strap 40 wherein there is a slot. The set of different lengths enables customization of the amount of mandibular advancement that may work most effectively for a particular user. Typically, the relative positions of pins 45 fixed on the mandibular denture 20 and maxillary denture 30 is such that use of shortest strap results in maximum mandibular advancement, whereas the use of longest strap results in minimum mandibular advancement. Different strap lengths may be tried between these to optimise the amount of mandibular advancement best suited for a particular user considering factors such as clearance of airway passage, and comfort of use including reduction or management of side effects such as temporomandibular joint (TMJ) pain. Optionally and preferably, the configuration of a strap 40 illustrated in this Figure has a slot recess connecting the pin holes to provide sliding between the pin and the slot thus better flexibility of movement between maxillary and mandibular jaws in forward-backward, upward-downward, and lateral (leftward-rightward) directions. Those skilled in the art may appreciate that there may be other number of straps in a set ranging from 2 to 10 or more or even a single strap optimised a priori before handing over to a user, and all such possibilities are within the scope and spirit of this invention disclosure.

Optionally, and in addition to the configurations described so far, FIG. 2C illustrates a circular, elliptical, and a rectangular portion of increased sized recess or slot portion at the middle portion of the strap. This larger recess in the slot of strap is intended to allow a circular, elliptical or rectangular head 46 of pin 45 to come out of the strap facilitating ease of change of strap. This configuration of the invention is incorporated to ameliorate the problem of unwanted disengagement between pin and strap when the pin hole or slot connecting the pin holes is kept too large to facilitate the ease of change of strap. By providing a larger recess only at the middle part, the disengagement during use inside mouth of the user is avoided. As the strap is required to be changed only outside the mouth of user, the maxillary and mandibular dentures can be freely moved to a relative position such that a pin can access the wider recess at the middle of a strap to facilitate removal of strap from pin. The figure shows increase in outer width of the strap to retain strength of the strap at the middle portion.

FIG. 2D illustrates the different shapes of pin heads 46, viz. circular, elliptical and rectangular cross-sections, as can be seen in end view-P, whereas, the pin is generally circular cross section in most cases.

An optional but additional configuration of the strap has a stronger reinforcement cord embedded inside a relatively weaker strap material to solve a problem that was encountered during trials, namely tearing off or severance of the strap. This is of particular relevance and advantage of this disclosure where the strap is made of flexible material, for example, made of one of the acceptable bio-compatible elastomers (Saliterman, Steven S., “Introductory Medical Device Prototyping: Medical Device Polymers”, Presentation, Dept. of Biomedical Engineering, University of Minnesota, USA); whereas the reinforcement cord may be made of a stronger material such as Kevlar® (DuPont®, headquartered at Wilmington, Delaware, USA, manufactured by Advanced Fibre Systems, VA, USA). FIG. 2E (not to scale, only indicative representation) shows a sectional view of a typical multi-layer strap configuration with a stronger cord material reinforcing as core to enable improved durability with flexibility. The circular cross section of the reinforcing cord 41 can be seen embedded inside the strap cross-section. The combination of both strength as well as flexibility allows the relative movement between the mandibular and maxillary parts of the denture in multiple directions of 3D space, viz. forward-backward, upward-downward, leftward-rightward. The multi-layer construction may be achieved by several alternative manufacturing means such as molding, 3D printing, or other methods that may be available in the state-of-the-art, gainfully deployed for this application.

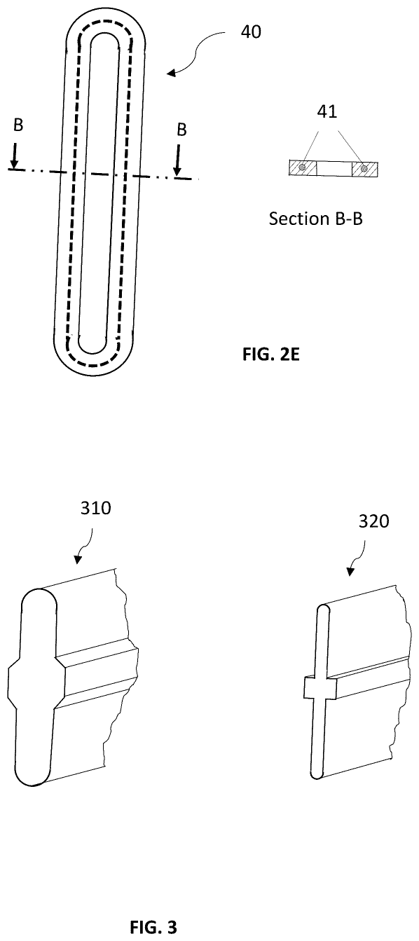

Mass production of straps deploying economy of manufacture scale may be an optional yet another additional method deployed as an alternative to a stronger reinforced strap configuration. FIG. 3 illustrates a perspective view of configuration of a typical punch set showing the contours of the end faces of the punches 310 and 320 that may be deployed for progressive 2-step blanking process, each using a different punch and die set (dies are not shown in the figure) as an optional manufacturing process for straps (for example, the third shape out of the shape options illustrated in FIG. 2C) to enable economy of scale, wherein a large number of inexpensive spare straps may be provided instead of stronger and costlier straps. It can be appreciated by those skilled in the art that a punch-die set comprises of closely slidably fitting cavity in a die that receives a correspondingly shaped protrusion known as punch, the relative impact between die and punch piercing the part of a sheet material of interest placed between a punch and a die. In the illustrated case, the pierced part produced by the first set of punch-die interaction will produce an interim part as blank, while the second set of punch and die interaction will produce a useful intended strap part and a scrap or waste part corresponding to the shape of slot or recess in the strap. We have found that bio-compatible versions of materials such as Kapton® (DuPont®, headquartered at Wilmington, Delaware, USA) available readily in sheet form are suitable raw material for such blanking process.

While the figures and description in this disclosure has presented in detail full maxillary and mandibular dentures, we have developed several additional and optional embodiments that are covered by the scope and spirit of the overall invention. For example, an embodiment of the invention has a typical RPD set comprising of a maxillary part, a mandibular part, an embrasure clasp, a pair of multiple circlet clasps, and a pair of straps connecting the maxillary and mandibular parts to enable desired mandibular advancement. Clasps are means to secure partial dentures with the help of surviving natural teeth in a person's jaws. The terminology of different types of clasps are well known in the denture related art, but to our knowledge not gainfully deployed or used in oral appliances for obstructive sleep apnea or anti-snoring devices. We have experienced and found the forces that can be withstood by unique designs and arrangements of different types of retainers/clasp assemblies, without glue, to have enough retention force to withstand mandibular advancement force that is in fact quite small. This is found to be valid for several clasp designs, for example, Circumferential (Circle or Akers) clasp, Ring clasp, Embrasure (Double Akers) Clasp, “C” clasp (Hair-pin or Reverse action), etc.

Another optional variant of the embodiment described above is the one wherein an improvised embrasure clasp is deployed to enable facilitation of resting the tongue of wearer constraining it so that it does not slide backwards contributing to airway blockage.

In yet another optional and additional variant of the embodiment discussed so far, a combination of multiple improvised clasps is deployed to enable facilitation of adjustable mandibular advancement with or without the need for straps.

Furthermore, in another optional variant of the embodiment presented above, a combination of multiple improvised clasps is deployed to enable facilitation of adjustable mandibular advancement without the need for straps, additionally providing for resting the tongue of wearer constraining it so that it does not slide backwards contributing to airway blockage.

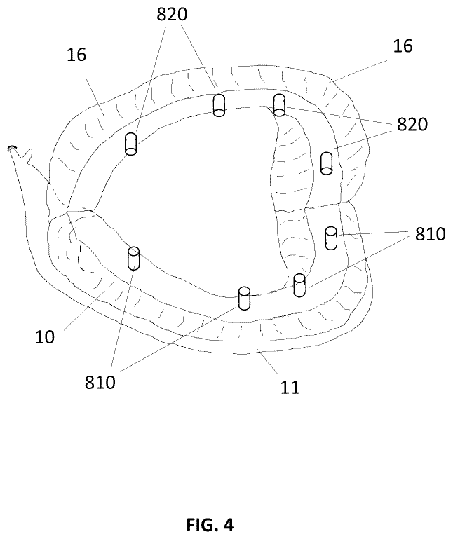

Furthermore, another optional embodiment of the invention has a typical ISD set comprising of a maxillary part, a mandibular part, and a pair of straps connecting the maxillary and mandibular parts to enable desired mandibular advancement. FIG. 4 illustrates typical locations of implant pins 810 on the mandible's lower gum 10 over bone structure 11, and implant pins 820 on the upper gum 16 of maxilla that are used to retain the denture on the wearer's jaws, essentially serving the purpose of retention of denture without the use of adhesive. The dentures possess mating holes that slidably and snugly receive corresponding implant pins to form good connection and thus retention of dentures over the gums. These dentures are found to have higher retention force capacity compared to adhesive based retention, thus can very easily overcome the mandibular advancement forces. Those skilled in the art can appreciate that the number of implants and their locations depend on several factors such as jawbone density, jawbone loss, bone grafting, etc.; and thus although 2 to 4 implants per jaw are typical, there may be more implants necessary in some cases, for example, 12 to 16 dental implants have been found necessary in some cases. For the purpose of withstanding the mandibular advancement force of approximately 1 to 1.2 Newtons per millimeter of mandibular advancement, considering that the advancement required is generally within 5 millimeters, these numbers of implants are adequate. Generally, the number of implants needed for the dual purpose of general denture retention for chewing and for mandibular advancement is found to be same or close to the number of implants needed for single conventional purpose of denture retention only for chewing.

Furthermore, in another optional but additional variant of the embodiment described above, an improvised embrasure clasp is deployed to enable facilitation of resting the tongue of wearer constraining it so that it does not slide backwards contributing to airway blockage.

Furthermore, in yet another optional but additional variant of the embodiment described above, a combination of multiple improvised clasps is deployed to enable facilitation of adjustable mandibular advancement with or without the need for straps.

Furthermore, in yet another optional but additional variant of the embodiment described above, a combination of multiple improvised clasps is deployed to enable facilitation of adjustable mandibular advancement without the need for straps, additionally providing for resting the tongue of wearer constraining it so that it does not slide backwards contributing to airway blockage.

Additionally, but optionally, a universal version of the invention utilizing a compliant clasp approach is also invented that may be worn over either denture or natural teeth due to its lightweight and ease of attachment-detachment configuration to facilitate anti-snoring provisions.

Furthermore, in yet another optional but additional variant of the embodiment described above, another universal version of the invention utilizes a compliant clasp approach enabling stepless mandibular adjustment that may be worn over either denture or natural teeth due to its lightweight and ease of attachment-detachment configuration to facilitate anti-snoring provisions.

Furthermore, yet another optional but additional universal version of the invention utilizes a compliant clasp approach that may also be elastic enabling stepless mandibular adjustment with spring characteristic that may be worn over either denture or natural teeth due to its lightweight and ease of attachment-detachment configuration to facilitate anti-snoring provisions with enhanced comfort for the wearer allowing jaw movement.

Furthermore, yet another optional but additional universal version of the invention utilizes a reactive latch mechanism that realizes a mandibular advancement only after the user has gone on sleep causing excessive relaxation of the mandible relative to the maxilla, to facilitate anti-snoring provisions with enhanced comfort for the wearer allowing natural or moderate relaxed jaw positions during onset of sleep.

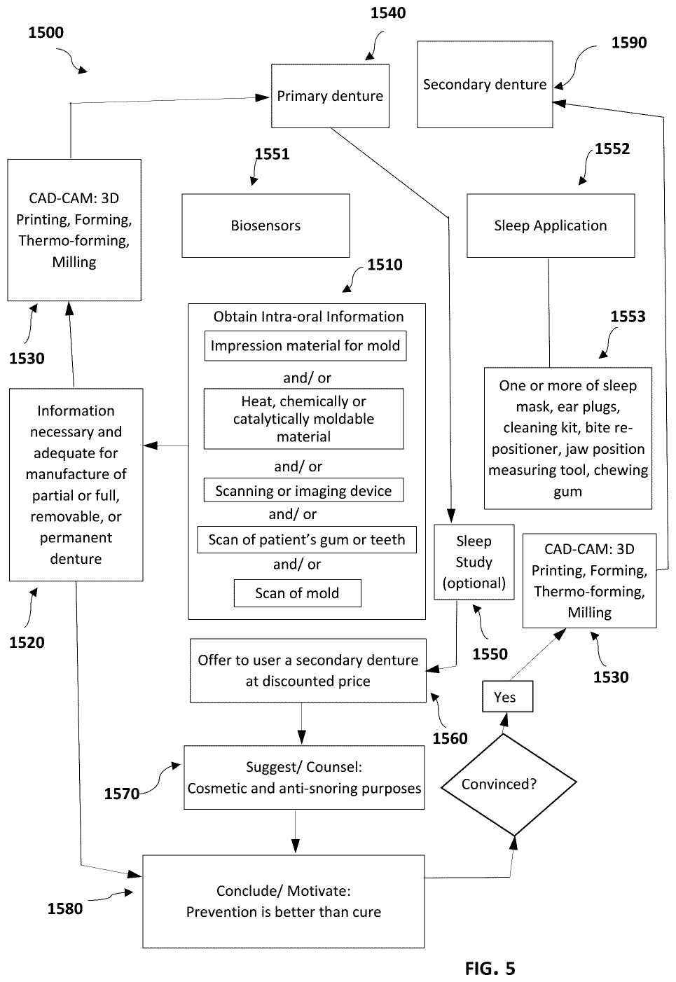

FIG. 5 illustrates the schematic diagram for an optional additional inventive process 1500 comprising of economical production of a secondary unit that may be offered to a patient ordering a primary partial or full, removable or permanent denture; at a discounted price that may serve the purpose of cosmetic function as well as anti-snoring function. This also illustrates gainful deployment of a business process promoting sale of primary denture, and/or add-on revenue generation through sale of secondary denture. This innovative process obtains intra-oral information 1510 that may involve use of either conventional mold material usage such as impression material and allied equipment for capturing the configurations of user's mandibular gum and maxillary gum or adjacent teeth in addition to the gums in the case of removable partial dentures (RPDs). Alternately, heat, chemically, or catalytically moldable materials may be used to obtain the impression. Optionally, other alternatives such as scanning of user's gum or teeth using imaging devices or scan of mold may be resorted to. This information 1520 is necessary and adequate for manufacture of partial or full, removable or permanent denture, and may be gainfully used or deployed for manufacturing either a primary denture 1540 and/or a secondary denture 1590. The manufacturing process 1530 may include Computer Aided Design (CAD) and Computer Aided Manufacturing (CAM) comprising of different options such as 3D printing, forming, thermo-forming such as injection molding, or milling.

While delivering the primary denture ordered by the user, or before the ordering process, or during the ordering process, or subsequently, an optional sleep study 1550 may be offered to the user. The sleep study may comprise of any of the different levels of sleep studies (Level 1, Level 2, Level 3, Level 4) either at a sleep lab or at home. With or without this optional sleep study, the user of denture may be offered a secondary denture at a discounted price. This offer process step 1560 involves suggestion and counselling 1570 about the advantages of a secondary set of dentures to be worn at night during sleep time that may be either an exact duplicate of the primary denture, or alternatively and preferably; a simpler, lighter, version with provisions such as increased exposure of gums to air as the purpose of this secondary denture is not chewing of food. While serving cosmetic purposes avoiding any embarrassing situations where the user maybe exposed to known or new acquaintances by getting caught off-guard without any denture, the secondary denture also serves the important purpose as an anti-snoring device. Even if the user is not aware or not formally diagnosed for sleep disorders knowingly or un-knowingly, the secondary denture serves an important function of prevention is better than cure motto 1580. Once the user is convinced of the advantages of acquiring a secondary denture at discounted price, the necessary information 1520 for production that was available earlier for primary denture production may be advantageously re-used for manufacture process 1530 to produce the secondary denture 1590. While sleep study 1550 is just optional, in case the same is preferred by a user, furthermore optionally, related additional appliances such as a variety of biosensors 1551, sleep application 1552, and a kit 1553 comprising of one or more of a sleep mask, ear plugs, cleaning kit, bite re-positioner, jaw position measuring tool, and chewing gum may be offered and deployed for the sleep study. The biosensors 1551 deployed may be one or more biosensors configured to measure one or more sleep parameters, wherein the one or more sleep parameters are selected from heart rate, breathing rate, head movement, body movement, bite force, body temperature, blood glucose levels, actigraphy, oximetry, apnea hypopnea index (AHI), peripheral arterial tone (PAT), cardiac data, chest expansion, respiratory air flow, sleeping position, patient compliance and snoring.

We claim:

1. A system for preventing or assisting in reducing condition of snoring or obstructive sleep apnea and method of use of the system for increased use of the system for anti-snoring and obstructive sleep apnea reducing by providing the system and steps of method comprising of
obtain intra-oral information necessary and adequate for manufacture of denture;
manufacture and provide primary denture set comprising of
a mandibular denture adapted to be worn on lower jaw of a user;
a maxillary denture adapted to be worn on upper jaw of the user;
suggest and offer a secondary denture set;
follow through consent of user to deploy earlier obtained intra-oral information for manufacture of a secondary denture set;
provide secondary denture set comprising of
a mandibular denture adapted to be worn on lower jaw of a user wherein retention of the denture on user's jaw is enhanced by use of glue between the denture and gum or by use of an implant on jaw or by use of a clamp or clasp between the denture and natural or implanted teeth;
a maxillary denture adapted to be worn on upper jaw of the user wherein retention of the denture on user's jaw is enhanced by use of glue between the denture and gum or by use of an implant on jaw or by use of a clamp or clasp between the denture and natural or implanted teeth;
a means to link together the mandibular and maxillary dentures for enabling lower jaw to advance relative to the upper jaw facilitating mandibular advancement.

2. A method of use of claim 1, wherein said secondary denture set is provided with different characteristics compared to the primary denture set adequate or more suitable for cosmetic or anti-snoring or both purposes.

3. A method of use of claim 2, wherein said different characteristics of secondary denture set is fully or partially simpler or lighter or with provision of increased exposure of gums to air or a combination of characteristics making it more suitable for wearing during sleep time compared to the primary denture meant for chewing of food.

4. A method of use of claim 1, wherein said secondary denture set for cosmetic or anti-snoring or both purposes is provided for a different price compared to the primary denture set.

5. The system and method of use of claim 1, wherein the means to link together the mandibular and maxillary dentures comprises of at least two pins and a strap or four pins and a pair of straps.

6. The system and method of use of claim 5, comprising of a set of straps wherein each pair of straps is of different length deployed to vary the amount of mandibular advancement.

7. The system and method of use of claim 5, wherein the strap or straps have a recess to allow relative movement between the mandibular and maxillary dentures.

8. The system and method of use of claim 1, wherein said mandibular and maxillary dentures constitute a denture set that is provided as removable partial dentures or removable full dentures.

9. The system and method of use of claim 1, wherein said mandibular and maxillary denture set is provided as any one of implant supported dentures, namely single tooth dental implant, or multiple-tooth on implant-supported bridge, or multiple-tooth on implant-retained denture.

10. The system and method of use of claim 1, wherein one or more parts of the system, namely mandibular denture, maxillary denture, and means to link them together are made of bio-compatible Nylon.

11. The system and method of use of claim 1, wherein at least one or more parts of the system, namely mandibular denture, maxillary denture, and means to link them together are made by 3D printing or additive manufacturing process.

12. The system and method of use of claim 1, wherein all parts of the system, namely mandibular denture, maxillary denture, and means to link them together are made by 3D printing or additive manufacturing process.

13. The system and method of use of claim 5, wherein the strap or straps are made from a blanking process using a punch and die set.

14. The system and method of use of claim 5, wherein the strap or straps are made of multi-layer hybrid construction wherein a material such as Kevlar® cord is core to provide strength over which bio-compatible material such as Nylon-12 is coated allowing a combination of both strength as well as flexibility that facilitates relative movement between the mandibular and maxillary dentures in multiple directions of 3D space, specifically forward-backward, upward-downward, leftward-rightward, or other possible intermediate directions.

15. The system and method of use of claim 8, wherein retention of mandibular or maxillary partial denture on user's jaw is enhanced by use of at least one clamp or clasp between the partial denture and one or more natural teeth.

16. The system and method of use of claim 1, wherein at least one improvised clasp or a combination of multiple improvised clasps comprising of at least one adaptation of circumferential (circle, circlet or akers) clasp, ring clasp, embrasure (double akers) clasp, “C” clasp (hair-pin or reverse action) is deployed.

17. The system and method of use of claim 16, wherein at least one improvised clasp is made of a compliant material configured for resting and constraining tongue of wearer.

18. The system and method of use of claim 16, wherein the system may be worn partially over jaw and partially over natural teeth, the system comprising of a mandibular removable partial denture adapted to be worn on a part of lower jaw of a user;
a maxillary removable partial denture adapted to be worn on a part of upper jaw of the user;
a means to hold the mandibular and maxillary partial dentures on the jaws of the user by anchoring around at least one natural tooth;
a means to link together the mandibular and maxillary partial dentures or their extended embodiments holding them on respective jaws, for enabling lower jaw to advance relative to the upper jaw facilitating mandibular advancement.

19. A method of use of claim 1, wherein a sleep study is offered during suggesting and offering the secondary denture set to increase the likelihood of obtaining follow through consent of user to deploy earlier obtained intra-oral information for manufacture of a secondary denture set.

20. A method of use of claim 19, wherein said sleep study is offered by providing one or more of biosensors configured to measure one or more sleep parameters, wherein the one or more sleep parameters are selected from heart rate, breathing rate, head movement, body movement, bite force, body temperature, blood glucose levels, actigraphy, oximetry, apnea hypopnea index (AHI), peripheral arterial tone (PAT), cardiac data, chest expansion, respiratory air flow, sleeping position, patient compliance and snoring, and in addition a sleep application, and kit comprising of one or more of sleep mask, ear plugs, cleaning kit, bite-repositioner, jaw position measuring tool or chewing gum.
